(19) United States
(12) Patent Application Publication
(10) Pub. No.: US 2010/0240605 A1
(43) Pub. Date: Sep. 23, 2010

(54) NOVEL IMMUNOTHERAPY STRATEGY

(75) Inventors: Ghislain Opdenakker, Sint- Amandsberg (BE); Jozef Van Damme, Brussel (BE)

Correspondence Address: CLARK & ELBING LLP, 101 FEDERAL STREET, BOSTON, MA, 02110, US

(73) Assignee: KATHOLIEKE UNIVERSITEIT LEUVEN K.U. Leuven R&D, Leuven (BE)

(21) Appl. No.: 12/670,520

(22) PCT Filed: Jul. 28, 2008
(86) PCT No.: PCT/BE08/00060

§ 371 Date: Jan. 25, 2010

(30) Foreign Application Priority Data
Jul. 27, 2007 (GB) 0714650.9

(51) Int. Cl. A61K 31/715; A61P 37/00; A61P 35/00; G01N 33/53

(52) U.S. Cl. 514/54; 435/7.1

(57) ABSTRACT

The present invention relates to polyacetal-carboxylic acids, more in particular chlorite-oxidized oxyamylose (COAM) for use as a medicine, more in particular for the treatment or prevention of cancer and auto-immune disorders or relates to the use of polyacetal-carboxylic acids for the manufacture of a medicament for the prevention or treatment of cancer or auto-immune disorders. The invention provides also pharmaceutical compositions comprising said polyacetal-carboxylic acids and method of preventing or treating cancer and auto-immune disorders.

### FIELD OF THE INVENTION

The present invention relates to polyacetal-carboxylic acids, more in particular chlorite-oxidized oxyamylose (COAM) for use as a medicine, more in particular for the treatment or prevention of cancer and auto-immune disorders and relates to the use of polyacetal-carboxylic acids for the manufacture of a medicament for the prevention or treatment of cancer or auto-immune disorders. The invention provides also pharmaceutical compositions comprising said polyacetal-carboxylic acids and method of preventing or treating cancer and auto-immune disorders.

### BACKGROUND OF THE INVENTION

Despite the considerable research and financial resources which have been committed to the whole area of cancer, the burden of the disease is increasing, both in an epidemiological and a monetary sense. Despite earlier detection in many cancers, the mortality rate over the past 25 years has not decreased to any significant degree. Total incidence rates are increasing due to the general ageing of the population and due to the increasingly carcinogenic nature of our environments.

The main treatment strategies used for treating cancer are surgery, radiotherapy and chemotherapy. Today, also extracorporeal cell therapies are being applied. Extracorporeal cell therapies for cancer, vascular and inflammatory diseases are expensive, impractical and not compliant for patients and medical care givers. In these types of therapy cells are removed from the host, expanded or altered in vitro and reinjected into the patient with the aim of a clinical effect, e.g. tumor killing, neo-angiogenesis after infarction, substitution of cartilage cells in osteoarthritis (arthrosis), insulin-producing beta-cells for diabetes or substantia nigra cells for Parkinsonism, to name a few. Examples of extracorporeal therapies include lymphokine-activated killer therapy (LAK cells), hematopoietic and other types of stem cell therapy, tumor vaccination through antigen-activated lymphocytes or antigen-challenged dendritic cells. Some of these therapies are currently in experimental clinical studies, though often solid in vivo double-blind studies in experimental animal models are lacking. One invoked reason for failure of such therapies for cancer treatment is the absence of sufficient tumor antigenic stimulation. Other mechanisms include tumor dormancy and mechanisms of immune escape by tumors.

In any case, new cancer treatment options are highly needed, in first instance to decrease the mortality and secondly to decrease the cost associated with cancer prevention and treatment.

Autoimmunity is the failure of an organism to recognise its own constituent parts (down to the sub-moleculer level) as “self”, which results in an immune response against its own cells and tissues. Any disease that results from such an aberrant immune response is termed an auto-immune disease, the prominent examples being Systemic Lupus Erythematosus (SLE), Sjögren's syndrome and Rheumatoid Arthritis (RA). Autoimmune diseases are broadly classified into two categories, organ-specific and systemic diseases. The precise etiology of systemic auto-immune diseases is not identified. In contrast, organ-specific auto-immune diseases are related to a specific immune response including B and T cells, which targets the organ and thereby induces and maintains a chronic state of local inflammation. Examples of organ-specific auto-immune diseases include type 1 diabetes, myasthenia gravis, thyroiditis and multiple sclerosis. It is well recognized that suppression of this organ-specific immune response is beneficial and leads to partial or complete recovery of organ function in some instances. This kind of therapy is however not useful for all organ-specific autoimmune disease and furthermore, there is no therapy, which would suppress such an immune response in an antigen-specific manner. Current therapy rather makes use of non-specific suppression obtained by the use of corticosteroids and immunosuppressive agents, all exhibiting significant side-effects related to their absence of specificity, thereby limiting their use and their overall efficacy. All present treatments for autoimmune diseases are generally immunosuppressive, leading to side-effects of immunosuppression, i.e. an increased susceptibility to infections. Interestingly, for reasons that are far from being understood, the incidence of auto-immune diseases has doubled over the last 20 years, much in parallel to the increase observed in allergic diseases. Again, the cost related to the treatment of auto-immune diseases has increased enormously in recent years, adding a further argument to the need for a new form of therapy.

The prior art describes many therapies for cancer or auto-immune disorders, but does not lead a person skilled in the art to the present invention. More in particular, the prior art describes chlorite-oxidized oxyamylose (COAM) and its effect on Moloney Sarcoma Virus-induced tumor formation in mice after IP administration (E. De Clercq et al., Europ. J. Cancer 1972, Vol. 8, 535-540). This treatment did however not show a regression of tumors. The prior art also describes the use of COAM for the prevention of oncogenic virus induced cancer in C3H mice such as for the prevention of “spontaneously-occurring” carcinomas (which can also be ascribed to the presence of oncogenic viruses), after IP administration (A. Billiau et al, Life Sciences 1971, Vol 10, Part II, 643-647). The prior art therefore predominantly describes the mechanism of this effect against oncogenic virus induced tumors as being directed against the oncogenic viruses (e.g. preventing passage of virus, viral spread or virus multiplication).

The present invention relates to the treatment of auto-immune disorders and all types of cancer (excluding a direct anti-viral effect and thereby treating or preventing cancers other than those induced by oncogenic viruses) by using polyacetal-carboxylic acids such as COAM. The present invention circumvents a number of the problems encountered in cancer (immuno)therapy and therapy for auto-immune disorders and provides an alternative therapy for these diseases, in particular potentially with a higher activity (e.g. regression of cancer) and less side-effects. In particular, the present invention is the first example of an immunostimulating agent, that with a specific mode of application, can protect a host against autoimmune disease.

### SUMMARY OF THE INVENTION

The current invention describes the anti-cancer and auto-immune disorder modulating properties of polyacetal-carboxylic acids, more in particular chlorite-oxidized oxyamylose (COAM).

The present invention describes more in particular that polyacetal-carboxylic acids, with COAM as representative, recruit leukocytes, bind chemokines, protects normal hosts against lethal syngeneic tumors and protect against and treat cancer and EAE.

One aspect of the present invention relates to the use of polyacetal-carboxylic acids, more in particular chlorite-oxidized oxyamylose (COAM) as a medicine, more in particular for the treatment or prevention of cancer or auto-immune disorders. The present invention relates therefore to the use of polyacetal-carboxylic acids, more in particular chlorite-oxidized oxyamylose (COAM) for the manufacture of a medicament for the prevention or treatment of cancer and auto-immune disorders. The present invention thus relates to polyacetal-carboxylic acids, more in particular chlorite-oxidized oxyamylose (COAM) for use as a medicament, more in particular for the prevention or treatment of cancer or auto-immune disorders.

A second aspect of the invention relates to pharmaceutical compositions comprising polyacetal-carboxylic acids, more in particular chlorite-oxidized oxyamylose (COAM) and a pharmaceutically acceptable carrier, for use as a medicine, more in particular for the prevention or treatment of cancer or auto-immune disorders. In a particular embodiment, the pharmaceutical composition further comprises at least one other chemotherapeutic agent. In yet another particular embodiment, the pharmaceutical composition is in the form of an injectionable formulation, more particularly for intra-tumoral injection.

Another aspect of the invention relates to methods of preventing or treating cancer or auto-immune disorders by using polyacetal-carboxylic acids, more in particular chlorite-oxidized oxyamylose (COAM). In a particular embodiment, the invention relates to methods of preventing or treating cancer by intra-tumoral injection of polyacetal-carboxylic acids, more in particular chlorite-oxidized oxyamylose (COAM).

In particular embodiments, the cancers that can be treated by polyacetal-carboxylic acids, more in particular chlorite-oxidized oxyamylose (COAM) are selected from prostate, lung, breast, rectal, bladder, kidney, pancreatic, liver, ovarian, brain, uterine, skin or stomach tumors. More in particular, the cancers that can be treated by polyacetal-carboxylic acids, more in particular chlorite-oxidized oxyamylose (COAM) are solid cancers.

In another particular embodiment of the present invention, the cancers that can be treated are not (directly) related to a viral inducer of cancer such as for humans Human papilloma virus (HPV) for cervical cancer, Hepatitis B or C virus for liver cancer and the human T cell leukaemia virus for T cell leukaemia, or for mice and the like such as murine sarcoma virus (MSV). In a particular embodiment, the cancers to be treated by the present invention are selected from non-virus induced cancers or cancers not induced by oncogenic viruses and therefore exclude virus induced cervical cancer, viral induced liver cancer and viral induced T cell leukaemia in humans.

In another particular embodiment, COAM that is used for the treatment of cancers is administered intra-tumorally.

In particular embodiments, the auto-immune disorders that can be treated by polyacetal-carboxylic acids, more in particular chlorite-oxidized oxyamylose (COAM) are selected from autoimmune encephalomyelitis, multiple sclerosis, (type 1) diabetes, Systemic Lupus Erythematosus (SLE), Sjögren's syndrome and Rheumatoid Arthritis (RA), myasthenia gravis, thyroiditis, adrenalitis, polyendocrine syndromes, gastritis & pernicious anemia, ocular diseases, inner ear diseases, celiac disease, inflammatory bowel diseases and atherosclerosis.

The present invention shows that COAM can be used for cell deviation therapies. COAM can be used for applications of “Negative Endogenous Cell Therapy” (NECT) or “Positive Endogenous Cell Therapy” (PECT), whereby local (e.g. tissue-specific) depletion or accumulation respectively of cell populations (especially immune related cell populations like leukocytes, neutrophils, dendritic cells, macrophages or lymphocytes) is beneficial, such as in auto-immune related disorders or cancer respectively. The present invention therefore relates to a method for cell deviation therapy, negative endogenous cell therapy or positive endogenous cell therapy by using COAM.

Another aspect of the present invention relates to a method for the purification, binding or detection of chemokines by using COAM. In a particular embodiment, the method comprises the steps of contacting a (natural or unnatural) mixture of chemokines (e.g. plasma, serum, blood, urine, fractionated human samples, etc.) with COAM (whether or not on a carrier or in a column). In a particular embodiment, the method also comprises the step of washing the COAM contacted with chemokines with a solution which does not influence the binding between COAM and chemokines. In another embodiment, this step is followed by an eluting step wherein the chemokines are eluted from the COAM, e.g. by using a solution that disturbs the binding between COAM and the chemokines. In a particular embodiment, the present invention relates to a method for detecting chemokines in samples, said method comprising the step of contacting a sample with COAM, washing COAM with a solution which does not influence the binding between COAM and chemokines and detecting the chemokines (e.g. by using antibodies).

Another aspect of the present invention relates to high MW COAM, which is COAM which has the same or similar elution time in column fractionation as proteins of between 670.000 and 1000 Da. In another embodiment, high MW COAM is COAM obtained by collecting the fractionation volume eluted which contains the fractions with higher MW than vitamin B12 (1,353 Da) or which elutes proteins of higher MW than around 1000 Da in a column fractionation, more in particular in the volume eluting the protein fractions of MW of between 670.000 and 1000 Da. In a particular embodiment, the column fractionation is performed on a Hiload 16/60 Superdex 200 column (GE Healthcare), more in particular in 0.1 M NH4HCO3 pH 7.8, yet more in particular at a flow rate of 0.5 ml/min. In a particular embodiment, the high MW COAM elutes between 670 kDa and 1000 Da of protein markers, more in particular on a Hiload 16/60 Superdex 200 column (GE Healthcare), yet more in particular in 0.1 M NH4HCO3 pH 7.8 at a flow rate of 0.5 ml/min. Alternatively, the high MW COAM is the COAM which is eluted between 50 and 120 mL in a fractionation of COAM (in particular of around 500 mg to 1g) wherein COAM is fractionated on a Hiload 16/60 Superdex 200 column (GE Healthcare) in 0.1 M NH4HCO3 pH 7.8 at a flow rate of 0.5 ml/min.

In a particular embodiment of the invention, the COAM that is used in the present invention is “high MW COAM”. Thus, another aspect of the present invention relates to the use of high MW COAM as a medicine or to the use of high MW COAM as described herein for COAM or relates to pharmaceutical compositions comprising high MW COAM.

### BRIEF DESCRIPTION OF THE FIGURES

The examples and description of the invention, not intended to limit the invention to specific embodiments described, may be understood in conjunction with the accompanying Figures, incorporated herein by reference, in which:

FIG. 1: Fractionation of COAM on Superdex to obtain the fraction used in the examples.

Top chromatogram shows the separation of synthetic COAM. Arrows indicate the relative positions of the elution patterns of proteins with known molecular masses (indicated in Daltons). The indicated hatched part of the chromatogram was collected and rechromatographed and this is shown in the bottom chromatography.

FIG. 2: COAM binds mouse granulocyte chemotactic protein-2 (mGCP-2)

Upper chromatogram shows the elution pattern of high molecular weight COAM. Middle chromatogram shows elution of mouse GCP-2. The COAM preparation was incubated with 50 nanogram mouse GCP-2 and the mixture was analysed by gel filtration chromatography (lower figure) and the presence of GCP-2 in the column fractions was analysed with a sensitive ELISA for mouse GCP-2. It is clear that GCP-2 binds to COAM in solution and the complex comigrates at high molecular weight with COAM.

FIG. 3: The FIGS. 3 A, B, C and D show the results at the end of the experiments described in example 4 (protection of mice against lethal syngeneic tumors). At day 18, all tumors were excised and their volumes and weights were measured. These data are represented as volumes and weights of all tumors and as volumes and weights of not ulcerated tumors only.
3 A represents the volume in mm³ of the tumors including the “open” tumors;
3 B represents the volume in mm³ of the tumors excluding the “open” tumors;
3 C represents the weight in g of the tumors including the “open” tumors;
3 D represents the weight in g of the tumors excluding the “open” tumors;

FIG. 4 shows the results from a typical experiment with 5 saline control mice and 6 COAM-treated mice with syngeneic tumors. A significant reduction of tumor growth is observed after intratumoral COAM injection with a single dose of 100 μg COAM at Day 1 versus saline control.

FIG. 5: The FIGS. 5 A, B, C and D show the results of the comparison of COAM and heparin for their anti-cancer effect on syngeneic tumors in mice.
5 A shows the tumor volume including “open” tumors in function of time after injection of COAM, Heparin or saline;
5 B shows the tumor volume excluding “open” tumors in function of time after injection of COAM, Heparin or saline;
5 C shows the tumor volume including “open” tumors at day 18 after injection of COAM, Heparin or saline;
5 D shows the tumor volume excluding “open” tumors at day 18 after injection of COAM, Heparin or saline.

FIG. 6: Comparison of the effect of saline, COAM and heparin on the spleen weight.

FIG. 7: Protection by COAM against B16 melanoma formation in immunocompromised SCID mice.

FIG. 8: Protection by COAM against B16 melanoma formation in immunocompromised SCID mice.
8 A shows the mean tumor volume, while 8 B shows the mean tumor weight.

FIG. 9: Mobilization of neutrophils into the peritoneal cavity in response to COAM. Mice were immunized with SCH in CFA and treated with COAM (2 mg) or saline on days 0 and 7. Peritoneal cells were collected on days 5, 9 and 14, or 16 post immunization. (A) Morphological comparison of the composition of peritoneal cells between control mice and COAM-treated mice revealed the specific infiltration of neutrophils and a decrease in mast cells. Data represent three individual experiments taken together; bars represent averages±standard error of the mean of seven or nine mice. (B) Flow cytometric analysis of peritoneal cells of control mice and COAM-treated mice. Cells were stained for the presence of CD11b using FITC-labeled antibodies, and of Gr-1 using PE-labeled antibodies. Numbers indicated on each graph represent percent CD11b⁺Gr-1⁺ cells. One representative analysis out of 9 is shown.

FIG. 10: Neutrophil infiltration in the spinal cord and brain of COAM-treated mice is reduced compared with saline-treated control mice. (A) Morphologic examination of cytospin preparations stained with Hemacolor reveals a lower percentage of neutrophils in the brain and spinal cord of mice on day 16 post immunization, after treatment with COAM. Data of 3 pooled experiments are shown. Histograms represent averages±SEM. (B) Flow cytometry of cells isolated from the spinal cord and brain on day 16 post immunization of mice treated with COAM (2 mg) or saline on days 0 and 7. Cells were stained for CD11b and Gr-1. Numbers indicated on each graph represent percent CD11b⁺Gr-1⁺ cells. Each plot represents a staining pattern of pooled cells from 3 mice. Results are representative of three independent experiments with similar results.

FIG. 11: The clinical course of active EAE is attenuated in COAM-treated mice. SJL/J mice were immunized subcutaneously with syngeneic spinal cord homogenate for induction of EAE. They were treated i.p. with 2 mg COAM (filled boxes) or saline (open boxes) on day 0 (A), day 8 (B) or days 0 and 7 (C) post immunization. The clinical course of EAE in these mice is shown as mean daily group score±sem. The disease parameters are given as an insert. Dead animals were scored 6 from the day of death until the end of the study. Arrows point to the days of COAM administration. *p<0.05; **p<0.01 for comparison with saline-treated group; Wilcoxon test or Chi-square test.

FIG. 12: Gel filtration chromatography of COAM. (A) Analytical fractionation. Fifty mg of the COAM mixture was separated by gel filtration chromatography on a Superdex 200 10/300 GL column. After fractionation of COAM, pooled high molecular weight (MW) fractions were applied and eluted on the same gel matrix. This resulted in the expected similar elution position, which implies the chemical stability of the fractions. The hatched area indicates the pooled fractions. (B) For preparative fractionation of COAM, 700 mg of the mixture was separated on a Hiload 16/60 Superdex 200 column. A rechromatography of pooled fractions eluted at the same positions (shown as hatched areas). Both columns were first equilibrated and calibrated with a mixture of protein standards. The elution positions of these proteins are indicated in Da as size references, ranging from thyroglobulin (699 kDa), ferritin (440 kDa), IgG (150 kDa), bovine serum albumin (67 kDa), ovalbumin (43 kDa), cytochrome c (13 kDa) to vitamin B12 (1,353 Da).

### DETAILED DESCRIPTION OF THE INVENTION

### Definitions

The term “autoimmune disease” or “autoimmune disorder” refers to diseases that result from an aberrant immune response against its own cells and tissues due to a failure of an organism to recognise its own constituent parts (down to the sub-moleculer level) as “self”. The group of diseases can be divided in two categories, organ-specific and systemic diseases. The prominent examples of systemic immune disorders are Systemic Lupus Erythematosus (SLE), Sjögren's syndrome and Rheumatoid Arthritis (RA). Organ-specific auto-immune diseases are related to a specific immune response including B and T cells, which targets the organ and thereby induces and maintains a chronic state of local inflammation. Examples of organ-specific auto-immune diseases include type 1 diabetes, myasthenia gravis, thyroiditis, multiple sclerosis, adrenalitis, polyendocrine syndromes, gastritis & pernicious anemia, ocular diseases, inner ear diseases, celiac disease, inflammatory bowel diseases, atherosclerosis and also includes auto-immune encephalomyelitis. The autoimmune disorders are thus directed to own cells or tissues and include a reaction to “auto-antigens”, meaning antigens (i.e. of proteins) that are own constituent parts of the specific mammalian organism. In this mechanism, auto-antigens are recognised by B- and/or T-cells which will install an immune reaction against said auto-antigen.

The term “cancer” or “tumor formation” refers to a mass of abnormal tissue that arises without obvious cause from pre-existing body cells, has no purposeful function, and is characterized by a tendency to autonomous and unrestrained growth. Examples of tumors or cancers envisaged in the context of the present invention include but are not limited to prostate, lung, breast, rectal, bladder, kidney, pancreatic, liver, ovarian, uterine, brain, skin, stomach or other tumors.

The term “solid tumor” refers to non-metastised cancers or benign cancers (for a detailed description see below).

### DESCRIPTION OF THE INVENTION

The current invention describes the anti-cancer and auto-immune disorder modulating properties of the polyacetal-carboxylic acids, more in particular chlorite-oxidized oxyamylose (COAM). These properties are exemplified by the results described in this invention, namely that polyacetal-carboxylic acids, with COAM as representative, recruit leukocytes, bind chemokines, protects normal hosts against lethal syngeneic tumors, and prevents or treats cancer and EAE.

Thus the present invention relates to the use of polyacetal-carboxylic acids, more in particular COAM, for the inhibition or reduction of cancer and auto-immune disorders and for the manufacture of a medicament for the prevention, reduction or treatment of cancer or auto-immune disorders in a mammal. The present invention also relates to a method for the treatment, reduction or prevention of cancer or auto-immune disorder in a mammal by using polyacetal-carboxylic acids, more in particular COAM.

The product used is chlorite-oxidized oxyamylose or COAM, as synthesized by Claes et al. (Claes P, Billiau A, De Clercq E, Desmyter J, Schonne E, Vanderhaeghe H, De Somer P. Polyacetal carboxylic acids: a new group of antiviral polyanions. J. Virol. 1970, 5: 313-320).

More in particular, the present invention provides for the in vivo use of products that bind endogenous host chemokines in the prevention or treatment of disorders such as cancer and auto-immune disorders, instead of the use of recombinant exogenous chemokines or cytokines and their antibodies. The present invention also shows that compounds can induce local activation of endogenous leukocytes, including neutrophils, natural killer cells, macrophages, dendritic cells and other leukocytes within the tumor, leading to enhanced tumor immunogenicity and rejection. Optimal stimulation of tumor immunity by maximally making use of endogenous host molecules is achieved in the present invention by using as an example COAM. Another advantage is that in such a way homeostatic and endogenous compensatory mechanisms are kept alive, minimizing side-effects. For tumor therapy we name this strategy “Positive Endogenous Cell Therapy” or PECT, since the positive recruitment of cells into tumors is beneficial for the host.

For cancer treatment, the invention deals with COAM which forces antigen presenting cells (dendritic cells, macrophages, B lymphocytes) into the tumor to maximize tumor antigen uptake, processing and presentation. This particular step is often blocked by evasion mechanisms of tumors. The used product induces a massive and broad spectrum recruitment of leukocytes in contrast to single chemokines and thus circumvents tumor evasion strategies.

The proof of principle is provided here by intratumoral injection of COAM. However, other ways of intratumoral localization of the active substance are possible and are included herein. More in particular, the active substance COAM may be coupled to antibodies or other targeting factors and may be injected intravenously. These product conjugates (e.g. antibodies or derivatives) may recognize tumor-specific or tumor-associated antigens for tumor targeting.

The present invention shows that COAM can treat tumor formation in mice in vivo (see examples).

Current research suggests that each tumor arises from a single cell that has been transformed by one or more events. Such events include the activation of oncogenes and the absence or inactivation of specific tumor-suppressor genes. These transformed cells can form small clones, initially co-opting normal host vessels, growing to only several millimeters in size before their supply of nutrients becomes limited. At this point, the tumor may lie dormant for prolonged periods (from months to years) until ultimately undergoing destruction by the immune system or switching to an angiogenic phenotype. This “switch” involves a shift in the local equilibrium between negative and positive endogenous regulators of angiogenesis. The tumor cells may achieve this shift in several ways, including the overexpression of angiogenic factors, the recruitment of host cells (such as macrophages) that can produce their own angiogenic factors, the mobilization of angiogenic proteins from the extracellular matrix (ECM), or a combination of these processes. If the production of proangiogenic factors is sufficiently robust, neighboring endothelial cells will be activated, leading to the sprouting of new capillaries.

Tumors are quite different from inflammatory or other swellings because the cells in tumors are abnormal in their appearance and other characteristics. Abnormal cells—the kind that generally make up tumors—differ from normal cells in having undergone one or more of the following alterations: (1) hypertrophy, or an increase in the size of individual cells; this feature is occasionally encountered in tumors but occurs commonly in other conditions; (2) hyperplasia or an increase in the number of cells within a given zone; in some instances it may constitute the only criterion of tumor formation; (3) anaplasia, or a regression of the physical characteristics of a cell toward a more primitive or undifferentiated type; this is an almost constant feature of malignant tumors, though it occurs in other instances both in health and in disease. In some instances the cells of a tumor are normal in appearance and are faithful reproductions of their parent types so that the differences between them and normal body cells are difficult to discern. Such tumors are also often benign. Other tumors are composed of cells that appear different from normal adult types in size, shape, and structure. They usually belong to tumors that are malignant. Such cells may be bizarre in form or be arranged in a distorted manner. In more extreme cases, the cells of malignant tumors are described as primitive, or undifferentiated, because they have lost the appearance and functions of the particular type of (normal) specialized cell that was their predecessor. As a rule, the less differentiated malignant tumor cells are, the more quickly that tumor may grow. Malignancy refers to the ability of a tumor to ultimately cause death. Any tumor, either benign or malignant in type, may produce death by local effects.

The common and more specific definition of malignancy implies an inherent tendency of the tumor's cells to metastasize (invade the body widely and become disseminated by subtle means) and eventually to kill the patient unless all the malignant cells can be eradicated. Metastasis is thus the outstanding characteristic of malignancy. Metastasis is the tendency of tumor cells to be carried from their site of origin by way of the circulatory system and other channels, which may eventually establish these cells in almost every tissue and organ of the body. The amount of new blood vessel growth can correlate with poor prognosis in several tumor types. Since the shedding of large numbers of tumor cells from the primary tumor may not begin until after the tumor has a sufficient network of blood vessels, angiogenesis may also correlate with metastatic potential. Destruction of the ECM is probably necessary to initiate the metastatic process. Microvessel density has been correlated with cancer invasion and metastasis in a number of human tumors including breast, prostate, lung, esophageal, colorectal, endometrial and cervical.

In contrast to malignant tumor cells, the cells of a benign tumor invariably remain in contact with each other in one solid mass centred on the site of origin (“solid tumors”). Because of the physical continuity of benign tumor cells, they may be removed completely by surgery if the location is suitable. But the dissemination of malignant cells, each one individually possessing (through cell division) the ability to give rise to new masses of cells (new tumors) in new and distant sites, precludes complete eradication by a single surgical procedure in all but the earliest period of growth. A benign tumor may undergo malignant transformation, but the cause of such change is unknown. It is also possible for a malignant tumor to remain quiescent, mimicking a benign one clinically, for a long time. All benign tumors tend to remain localized at the site of origin. Many benign tumors are encapsulated. The capsule consists of connective tissue derived from the structures immediately surrounding the tumor.

Well-encapsulated tumors are not anchored to their surrounding tissues. These benign tumors enlarge by accretion, pushing aside the adjacent tissues without involving them intimately.

Among the major types of benign tumors are the following: lipomas, which are composed of fat cells; angiomas, which are composed of blood or lymphatic vessels; osteomas, which arise from bone; chondromas, which arise from cartilage; and adenomas, which arise from glands. For malignant tumors, examples comprise carcinomas (occur in epithelial tissues, which cover the body (the skin) and line the inner cavitary structures of organs (such as the breast, the respiratory and gastrointestinal tracts, the endocrine glands, and the genitourinary system). Sarcomas develop in connective tissues, including fibrous tissues, adipose (fat) tissues, muscle, blood vessels, bone, and cartilage. A cancer can also develop in both epithelial and connective tissue and is called a carcinosarcoma. Cancers of the blood-forming tissues (such as leukemias and lymphomas), tumors of nerve tissues (including the brain), and melanoma (a cancer of the pigmented skin cells) are classified separately.

In a specific embodiment, the polyacetal-carboxylic acids, more in particular COAM are used for the prevention and/or treatment of cancer in combination with any other therapy for cancer such as irradiation, chemotherapy or surgery.

In addition, the present invention also shows that also auto-immune disorders can be modulated beneficially by this strategy, next to cancer. In autoimmune diseases, leukocytes cause damage by inflammation. Deviation of leukocyte pools to body parts from which the leukocytes can easily be removed, results in a specific form of depletion that is associated with beneficial effects against autoimmunity. The proof of concept for this “Negative Endogenous Cell Therapy” or NECT—in which depletion of cell populations is beneficial—is also provided here with the in vivo reduction of EAE.

### Administration

The polyacetal-carboxylic acids, more in particular COAM and their physiologically acceptable salts (hereafter collectively referred to as the active ingredients) may be administered by any route appropriate to the condition to be treated and appropriate for the compounds to be administered. Possible routes include regional (e.g. intratumoral), systemical, oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

Regional treatment, more specifically intratumoral injection is useful for treatment of cancers in specific organs in the patient, including, but not limited to primary liver cancer, brain and kidney cancer and liver metastases from colon/rectal cancer. Treatment can be accomplished by intraarterial infusion. A catheter can be surgically or angiographically implanted to direct treatment to the affected organ, a subcutaneous portal, connected to the catheter can be used for chronic treatment, or an implantable, refillable pump may also be employed.

Intra-peritoneal injection of polyacetal-carboxylic acids, more in particular COAM is also a preferred route of administration, next to intra-tumorally injection.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The compositions used in these therapies may also be in a variety of forms. These include, for example, solid, semi-solid, and liquid dosage forms, such as tablets, pills, powders, liquid solutions or suspension, liposomes, suppositories, injectable and infusible solutions. The preferred form depends on the intended mode of administration and therapeutic application. The compositions also preferably include conventional pharmaceutically acceptable carriers and adjuvants which are known to those of skill in the art and which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986). Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness.

Preferably, the compositions of the invention are in the form of a unit dose and will usually be administered to the patient one or more times a day. Polyacetal-carboxylic acids, more in particular COAM, may be administered to the patient in any pharmaceutically acceptable dosage form, including intravenous, intramuscular, intralesional, intraperitoneal or subcutaneous injection.

It should, of course, be understood that the compositions and methods of this invention may be used in combination with other therapies, once improvement of the patient's condition has occurred, a maintenance dose is administered if necessary. Subsequently, the dosage or the frequency of administration, or both, may be reduced, as a function of the symptoms, to a level at which the improved condition is retained. When the symptoms have been alleviated to the desired level, treatment should cease. Patients may, however, require intermittent treatment on a long-term basis upon any recurrence of disease symptoms. It should be understood that the examples and embodiments described herein are for illustrative purposes only and that various modifications or changes in light thereof will be suggested to persons skilled in the art and are to be included within the spirit of this application and the scope of the appended claims.

The polyacetal-carboxylic acids, more in particular COAM, can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound.

### EXAMPLES

We describe in the examples that
1. COAM recruits leukocytes;
2. COAM binds chemokines;
3. COAM protects and treats normal hosts against lethal syngeneic tumors;
4. COAM protects a host against cancer and treats or reduces cancer in a host;
5. COAM protects a host against EAE and treats or reduces a host of EAE.

### Example 1

### Preparation of COAM

a. COAM

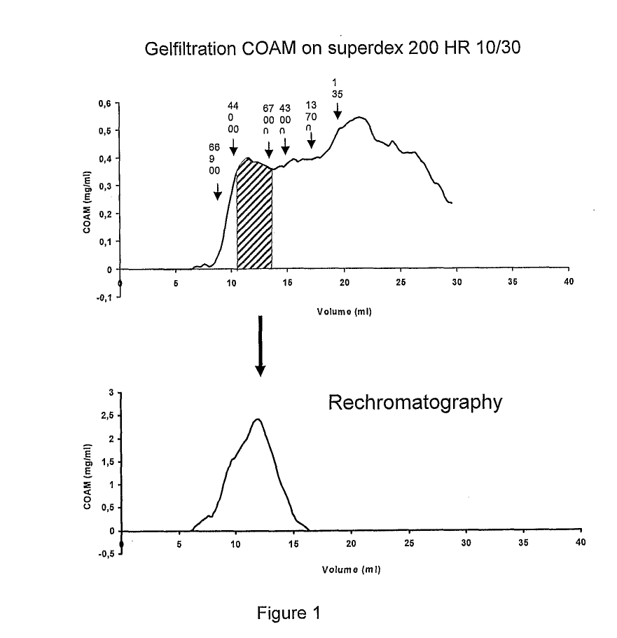

COAM is chlorite-oxidized oxyamylose, a polyacetal carboxylic acid, which is a polysaccharide-derived polyanion with a backbone consisting of —C—C—O—C—O— sequences with two attached carboxyl groups. COAM was synthesized by a two-step oxidation of amylose, as described previously according to Claes et al. J Virol 1970; 5:313-20. In the first oxidation step with periodate, cyclic monosaccharide structures were cleaved between two carbon atoms, yielding a polymer containing two aldehyde functions, called oxyamylose. The aldehyde functions are converted into carboxyl functions by oxidation with sodium chlorite. This product is a polymer with different lengths. COAM was separated by gel filtration chromatography in fractions with different polymer length (see FIG. 1). It is free of endotoxin and of protein contaminants. The endotoxin content of the product batches were determined by the Limulus amebocyte lysate test (Lonza, Verviers, Belgium). The COAM preparations were subjected to SDS-PAGE electrophoresis and any protein contamination was visualized with Coomassie Brilliant Blue R-250 (Sigma-Aldrich, St. Louis, Mo., USA) or with silver staining

(Silverquest™ Silver Staining Kit; Invitrogen). For mass spectrometry analysis, COAM (1 μg) was dissolved in 30 μl of 50% CH3CN/50% H2O/0.1% CH3COOH and was analyzed by electrospray ion-trap mass spectrometry in the negative mode with an Esquire_LC apparatus (Bruker, Bremen, Germany). Except where mentioned otherwise, all compounds were resuspended in sterile phosphate buffered saline (PBS; Lonza) for experimental use.

b. Fractionated COAM

We have fractioned synthetic COAM according to molecular weight by molecular sieving. For analytical fractionation, samples of 50 mg COAM from two different syntheses were applied on a Superdex 200 10/300 GL column (GE Healthcare, Chalfont St. Giles, UK) and equilibrated with 0.1 M NH4HCO3 pH 7.8 at a flow rate of 0.3 ml/min. Fractions of 0.4 ml were collected and, after lyophilization, the COAM content was quantified by weight. Subsequently, 8 fractions containing in total 7.9 mg high MW COAM were pooled and concentrated for rechromatography under the same conditions. Preparative fractionation of COAM was obtained with 700 mg preparation on a Hiload 16/60 Superdex 200 column (GE Healthcare) in 0.1 M NH4HCO3 pH 7.8 at a flow rate of 0.5 ml/min. Fractions of 0.4 ml were lyophilized and the COAM content was quantified. The columns were calibrated with a protein mix containing thyroglobulin (699 kDa), ferritin (440 kDa), IgG (150 kDa), bovine serum albumin (67 kDa), ovalbumin (43 kDa), cytochrome c (13 kDa) and vitamin B12 (1,353 Da), all purchased from Sigma-Aldrich. The sizes of the COAM variants (and fractions) were expressed in relative protein equivalent molecular weights. The reproducible separations were collected in three different fractions according to molecular weight (MW), corresponding to high MW, low MW, and very low MW COAM polymer fractions. Upon rechromatography, the selected high MW COAM preparation eluted at a similar retention volume as the pooled fraction. These observations showed that the fractions were chemically stable, that no interaction occurred between COAM and the column matrix and that the COAM polymers possessed a heterogenic MW.

When fractionated COAM is used in experiments, this is referred to with “high MW COAM”, “low MW COAM” or “very low MW COAM”.

### Example 2

### COAM Recruits Leukocytes

In a first set of in vivo experiments, 2 mg of COAM (unfractionated, solution of 2 mg in 100 μl sterile pyrogen-free 0.9% NaCl) was injected intraperitoneally per mouse and leukocytes were counted and defined at different times after injection of groups of 3-4 mice. Whereas control mice had on average 126.000 peritoneal cells of which the majority were peritoneal macrophages (and only 2.2% were neutrophils), within 2 hours these numbers increased to 630.000 cells (and 18.1% neutrophils) and at 4 h the numbers were 3.045.000 leukocytes (64.1% neutrophils) and, at 8 h, we measured 12.248.000 leukocytes (and 70.3% neutrophils). The mice survived without apparent morbidity. The test solution possessed not any toxic effect on various human and mouse cell lines.

The experiment was independently repeated in different groups of mice with similar results.

### Example 3

### COAM Binds Chemokines

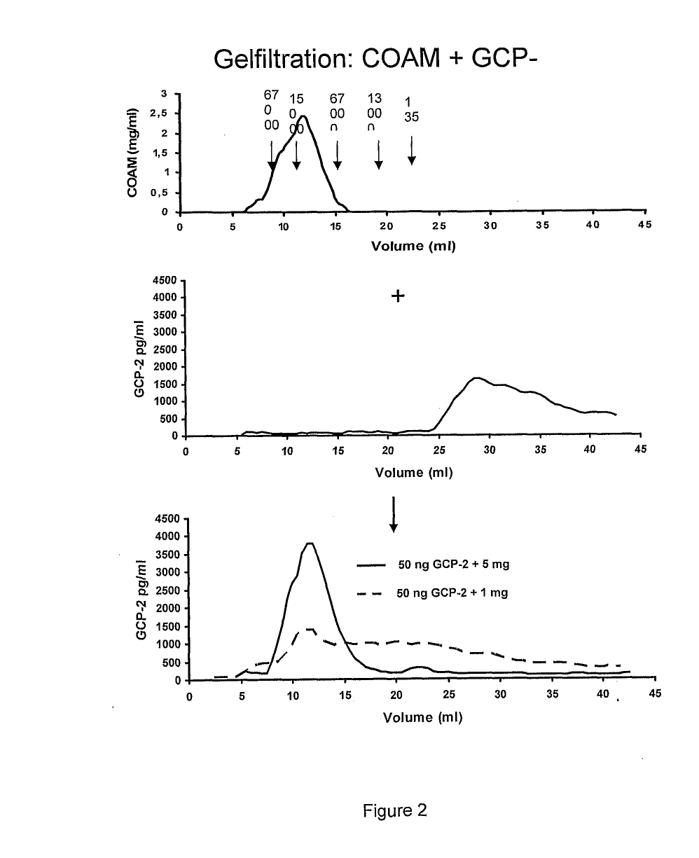

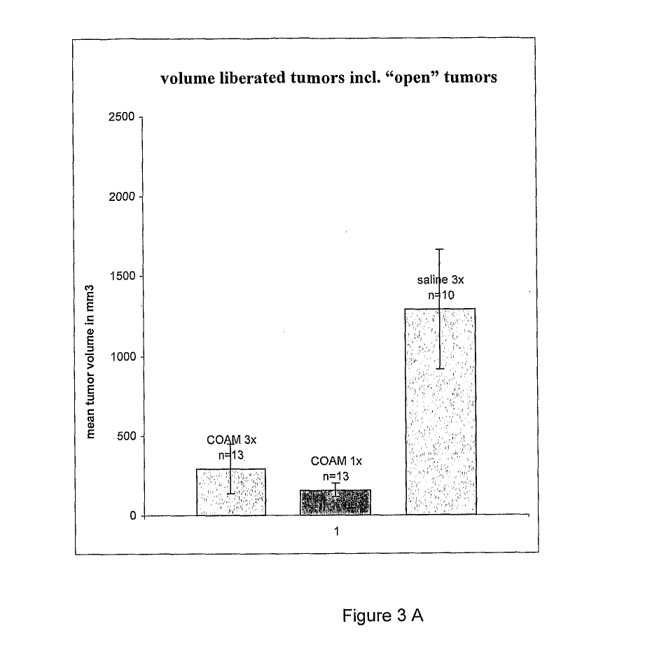

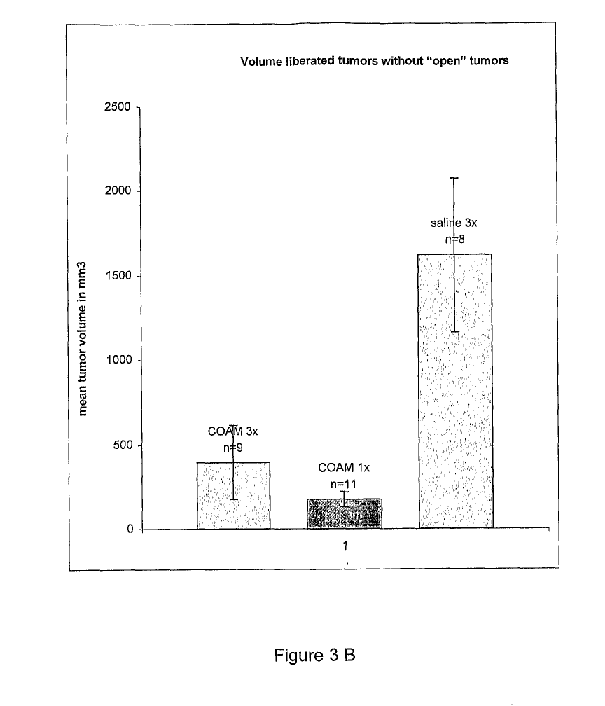

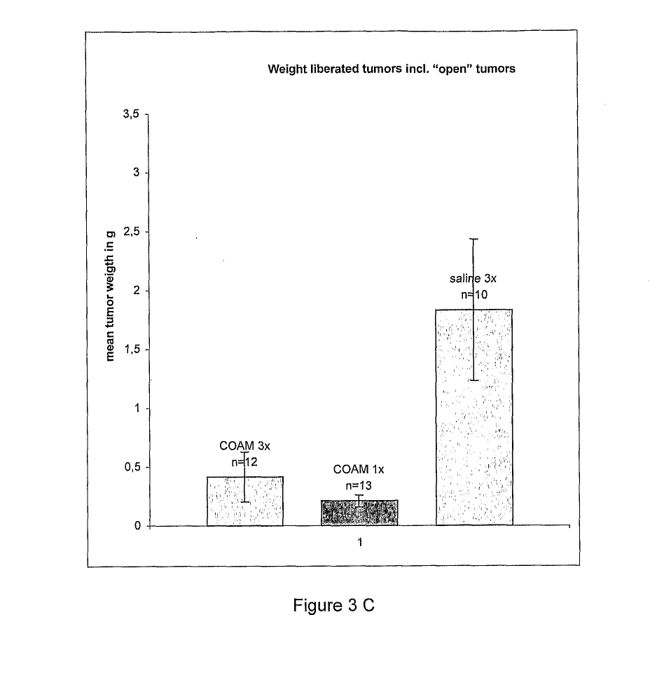

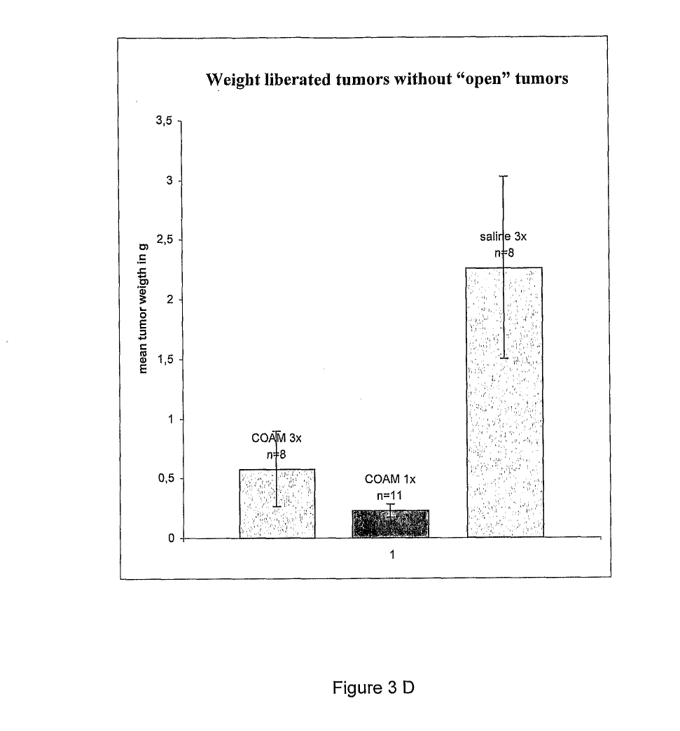

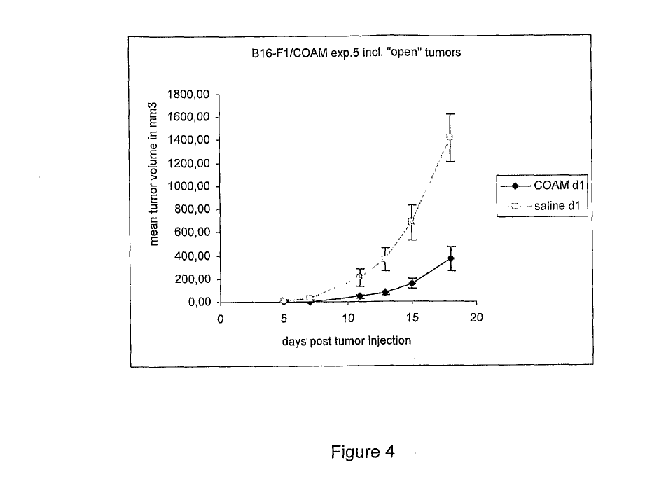

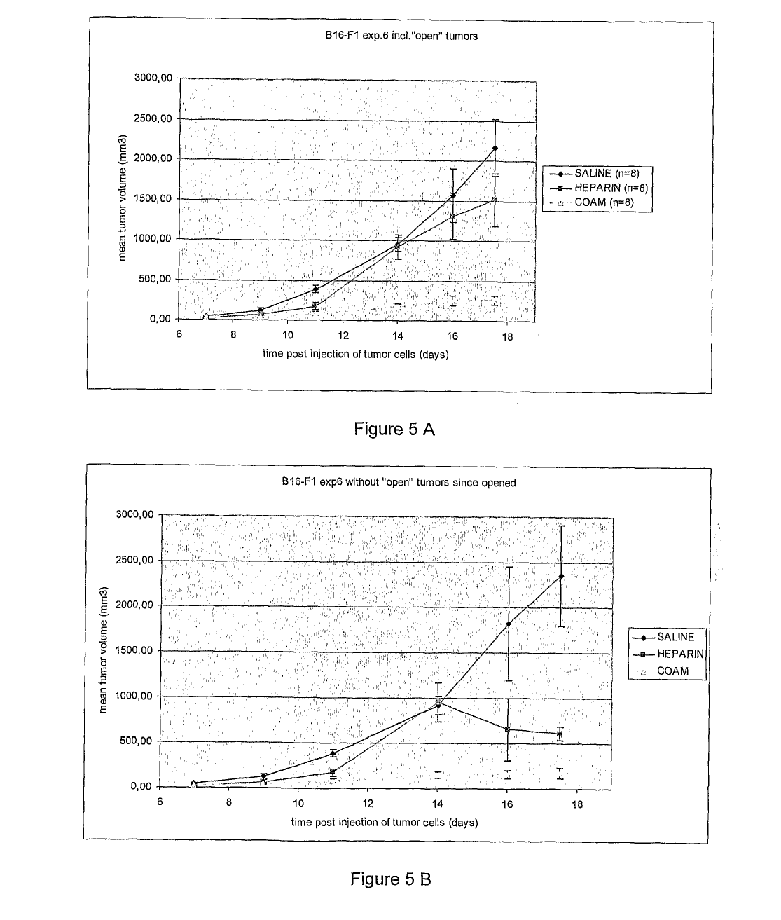

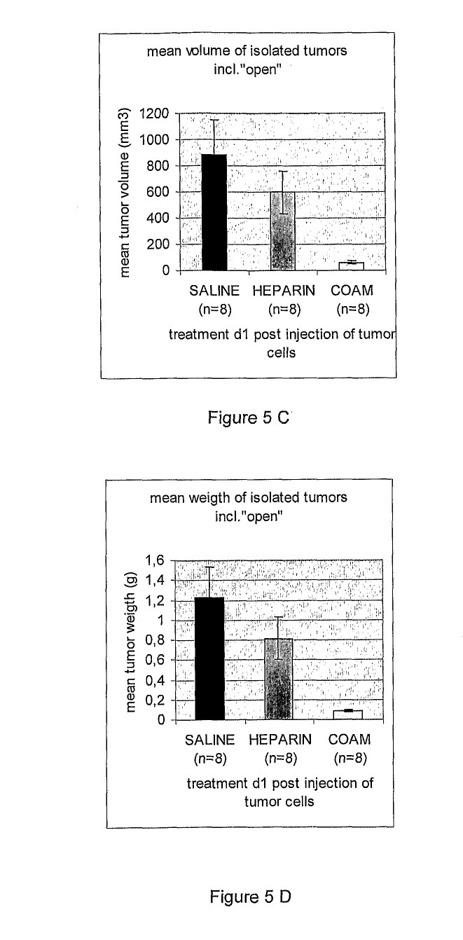

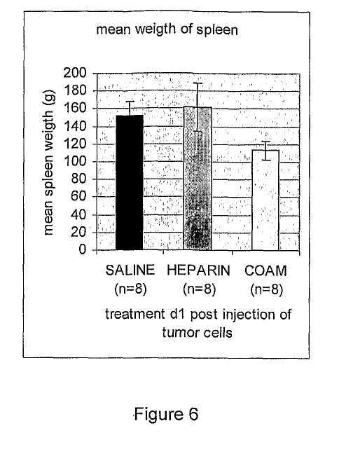

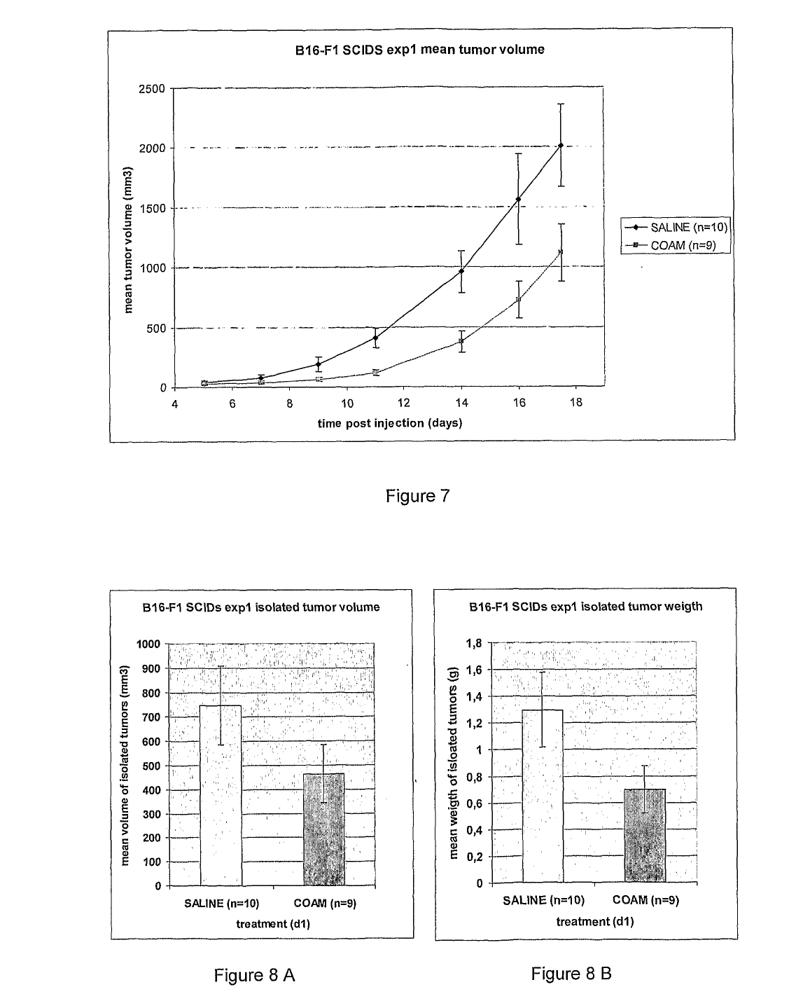

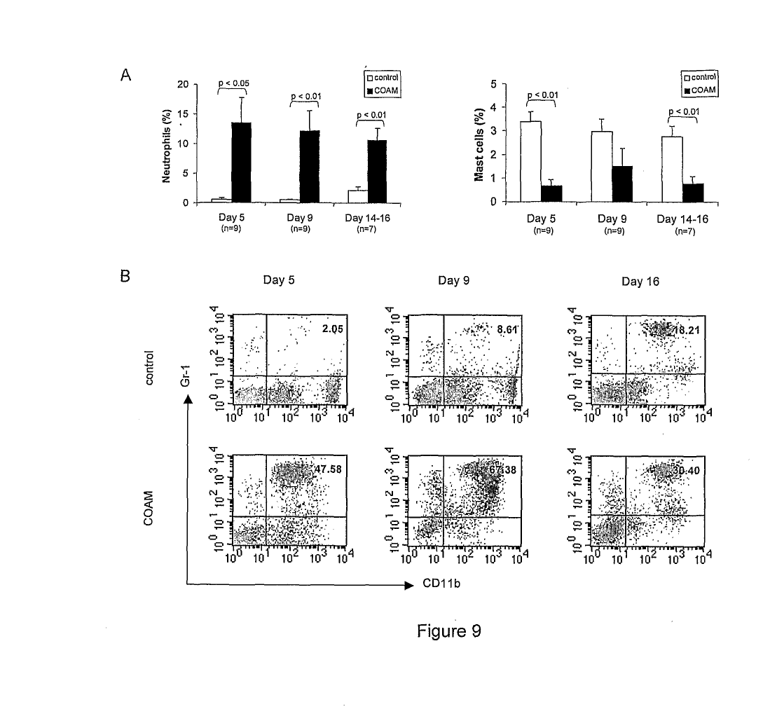

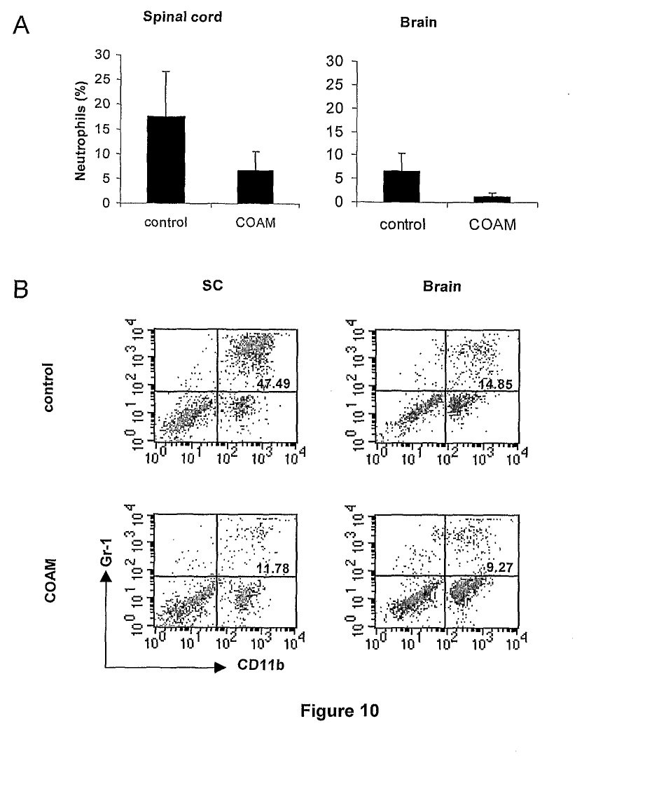

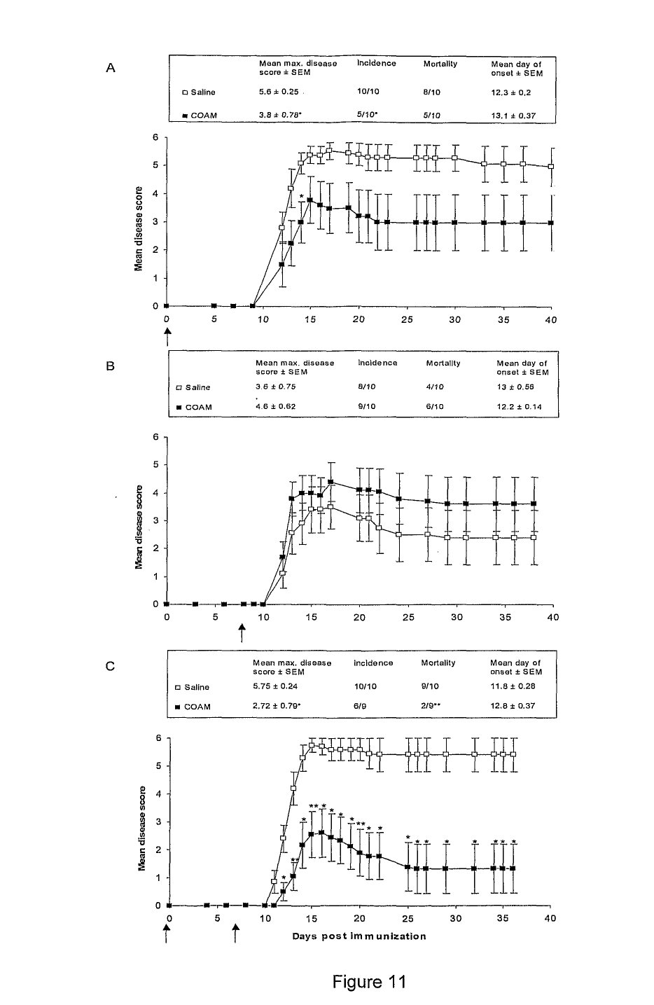

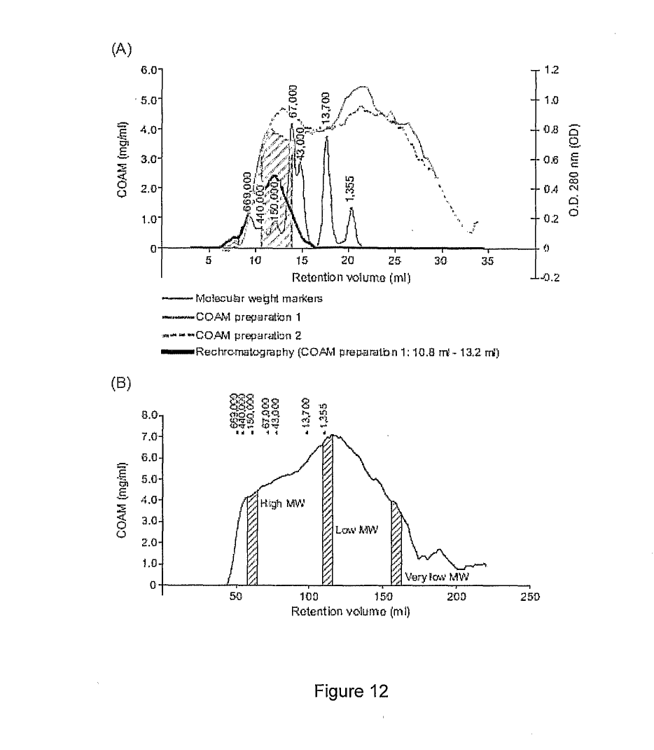

The binding by COAM of the most potent mouse neutrophil chemokine mGCP-2 was studied in solution by chromatographic procedures. COAM alone, mGCP-2 or COAM incubated in the presence of mGCP-2 were separated by classical gel filtration chromatography. The elution fractions were collected and the presence of mGCP-2 was determined in the elution fractions by ELISA specific for mGCP-2. Whereas GCP-2 alone eluted at the expected molecular mass of about 10 kilodaltons (positive control for ELISA) and COAM alone did not give any detection of mGCP-2 (negative control for ELISA), the solution of COAM together with mGCP-2 eluted in the high molecular weight range (larger than 50 kilodaltons) in the specific ELISA for mouse GCP-2. Since COAM itself is a mixture of polymers with different lengths, we fractioned first COAM in three fractions and then repeated the mGCP-2 binding and chromatographic analysis (as described above) with the high molecular weight COAM (high MW COAM) and mGCP-2. Again, mGCP-2 was found to be bound by high MW COAM. This is illustrated in FIG. 2.

In a second set of experiments human 500 ng interleukin-8/CXCL8 (IL-8/CXCL8) was tested. Again, in the presence of COAM, interleukin-8 coeluted with COAM at high relative molecular weight, whereas in the absence of COAM interleukin-8 eluted over a broad range of low molecular masses, corresponding to free human IL-8.

In a third set, 500 ng human monocyte chemotactic protein-1/CCL2 (MCP-1/CCL2) was tested. Again, free MCP-1 eluted at low molecular weight, compatible with the relative size of MCP-1. However in the presence of COAM, MCP-1 eluted at the position of the high molecular weight of COAM. This is again compatible with its binding to COAM

In a fourth example, we used synthetic human IP-10/CXCL10.

In view of these results with mGCP-2, IL-8, MCP-1, IP-10, we conclude that COAM is a general chemokine-binding factor and thereby propose the positive endogenous cell therapy with COAM mediating (natural or synthetic) chemokine binding.

### Example 4

### COAM Protects Mice Against Lethal Syngeneic Tumors

We used B16-F1 mouse melanoma tumors in C57BI6 mice. 0.5 million tumor cells were injected intradermally on day 0 as known in the art. In one experiment, control mice were injected with saline on days 1, 8 and 15. The treated group COAM d1 was injected intratumorally with a single dose of 100 μg COAM on day1. The treated group COAM d1, d8, d15 was injected with 100 μg COAM three times intratumorally. The overall mean tumor volume and weights as function of time were significantly reduced in the COAM-treated groups. In some cases the tumor ulcerated. With the exclusion of these ulcerated “open” tumors, also a significant effect on tumor burden was noticed. A clear and significant effect was already observed with a single intratumoral dosage of COAM. No additional effect was obtained when three injections instead of one injection of COAM were given. The FIGS. 3 A, B, C and D show the results at the end of these experiments (day 18).

At day 18, all tumors were excised and their volumes and weights were measured. These data are represented as volumes and weights of all tumors and as volumes and weights of not ulcerated tumors only.

FIG. 4 shows the results from a typical experiment with 5 saline control mice and 6 COAM-treated mice. A significant reduction of tumor growth is observed after intratumoral COAM injection. The effect of a single dose of 100 μg COAM at Day 1 versus saline control is clearly visible.

When several of such experiments were combined, the volume (p<0.015) and weight (p<0.025) were significantly reduced in the single COAM-treated group (COAM d1 only) compared to the saline control. As an additional note, the volume (p<0.030) and weight (p<0.05) were also significantly reduced in triple treated COAM versus control group.

Additional experiments were performed with essentially similar significant results. In particular, in the FIGS. 5 A, B, C and D, we used heparin as an additional control. To demonstrate that COAM has specific advantages above heparin, we compared heparin and COAM in an independent experiment. Heparin is a polyanion and binds chemokines and mimics heparin sulphate proteoglycans. The experiment was repeated as above with a single intratumoral dose of 100 μg COAM, 100 μg heparin and saline control at day 1 post tumor inoculation. Whereas heparin had some effect in the early phase, COAM had a lasting and significant effect on tumor expansion.

The spleen weight increases in most acute inflammatory conditions and can be used as a readout of systemic inflammation. As an extra control for general immune stimulation, the weights of the spleens were monitored. As demonstrated in FIG. 6, COAM did not induce a generalized systemic inflammatory effect since no overt splenomegaly was noticed.

### Example 5

### COAM Protects SCID Mice Against Human Cancer

In a next step we wanted to investigate whether an immunocompromised host might also benefit from COAM-treatment of cancer.

We used a similar protocol as above in immunocompromised SCID mice. The SCID mice received s.c. 500 000 B16-F1 cells on day 0. The results are indicated in the FIGS. 7 and 8 A and B

The results show that COAM also protects an immunocompromised host against tumors.

### Example 6

### COAM Protects a Host Against EAE

Experimental autoimmune encephalomyelitis was induced in mice (mice were immunized with SCH in CFA) and the groups were randomized. One group was treated with 2 mg COAM intraperitoneally on day 0 and day 7.

In FIG. 9 it is clear that COAM also in this situation resulted in increased neutrophil recruitment to the peritoneal cavity. Whereas neutrophils were increased by COAM, the mast cell population was reduced.

Table 1 shows the proportion of neutrophils in the peritoneal cavity of control and COAM-treated mice

CD11b⁺Gr-1⁺ cells (×10⁴).

| Day p.i. | Saline | COAM |
|---|---|---|
| 5 | 76.54 ± 32.18 (N = 9) | 322.78 ± 79.43 (N = 8) |
| 9 | 283.16 ± 61.73 (N = 8) | 1210.85 ± 272.05 (N = 9) * |
| 14-16 | 76.18 ± 23.39 (N = 6) | 276.66 ± 67.29 (N = 5) * |

Mice were immunized with SCH in CFA on day 0 and treated i.p. with 2 mg COAM or saline on day 0 and 7. Cells were obtained from the peritoneal cavity of saline- and COAM-treated mice on day 5, 9 and 14, 15 or 16 after immunization. Cells were stained with anti-CD11b-FITC and anti-Gr-1-PE antibodies. The proportion of CD11b⁺Gr-1⁺ cells is shown. Results represent pooled data from 3 experiments and N indicates the total number of mice examined. *p<0.05 between COAM-treated and saline-treated mice (Mann-Whitney U-test).

FIG. 10 also shows that neutrophil infiltration is reduced into spinal cord and brain after COAM injection into the peritoneum.

FIG. 11 shows that the clinical course of active EAE is attenuated in COAM-treated mice. SJL/J mice were immunized subcutaneously with syngeneic spinal cord homogenate for induction of EAE. They were treated i.p. with 2 mg COAM (filled boxes) or saline (open boxes) on day 0 (A), day 8 (B) or days 0 and 7 (C) post immunization. The clinical course of EAE in these mice is shown as mean daily group score±sem. The disease parameters are given as an insert. Dead animals were scored 6 from the day of death until the end of the study. Arrows point to the days of COAM administration. *p<0.05; **p<0.01 for comparison with saline-treated group; Wilcoxon test or Chi-square test.

As a conclusion, two injections of COAM protect a mammalian host against severe EAE by negative endogenous cell therapy or NECT.

### Example 7

### Effect of MW of COAM

Differences in activity between COAM fractions of varying polymeric length are compared. COAM fractions ranging from high to very low MW are compared with the unfractionated mixture of COAM. Fractions with high MW display a better activity in comparison with the mixture, while administration of COAM molecules of low MW are not different from the mixture, whereas very low MW COAM molecules are not contributing to the effect.

1-20. (canceled)

21. A method of preventing, reducing the severity or treating an auto-immune disorder or a cancer in a mammal, comprising the step of administering a polyacetal-carboxylic acid or derivatives thereof to said mammal.

22. The method according to claim 21, wherein said polyacetal-carboxylic acid is COAM (chlorite-oxidized oxyamylose).

23. The method according to claim 21, wherein said cancer is a primary tumor.

24. The method according to claim 21, wherein said cancer is a metastatic solitary or a multiple tumor.

25. The method according to claim 21, wherein said cancer is a solid cancer.

26. The method according to claim 21, wherein said cancer is a cancer which is not being induced by an oncogenic virus.

27. The method according to claim 21, wherein said step of administration is an intra-tumoral injection.

28. The method according to claim 21, wherein said autoimmune disorder is auto-immune encephalitis.

29. The method according to claim 21, wherein said step of administration is a systemic injection.

30. The method according to claim 22, wherein said COAM is a high molecular weight COAM.

31. The method according to claim 21, wherein said mammal is a human.

32. A pharmaceutical composition comprising a combination of a polyacetal-carboxylic acid or a derivative thereof with at least one chemotherapeutic agent, in a mixture with a pharmaceutically acceptable carrier.

33. The pharmaceutical composition according to claim 32, wherein said polyacetal-carboxylic acid is COAM (chlorite-oxidized oxyamylose).

34. A method for cell deviation therapy in a mammal comprising the step of administering to said mammal an effective amount of COAM.

35. A method for the purification, binding or detection of chemokines, the method comprising the steps of contacting a sample comprising chemokines with COAM and detecting the chemokines bound to COAM.
